(19) United States
(12) Patent Application Publication
(10) Pub. No.: US 2026/0128156 A1
(43) Pub. Date: May 7, 2026

(54) SYSTEMS FOR PROVIDING CONTEXT-BASED ASSISTANCE DURING AN ULTRASOUND IMAGING WORKFLOW

(71) Applicant: GE Precision Healthcare LLC, Waukesha, WI (US)

(72) Inventors: Svein Arne Aase, Trondheim (NO); Erik Normann Steen, Moss (NO)

(73) Assignee: GE Precision Healthcare LLC, Waukesha, WI (US)

(21) Appl. No.: 18/939,451

(22) Filed: Nov. 6, 2024

(51) Int. Cl. G16H 30/20; A61B 8/00; G06F 3/0482

(52) U.S. Cl. CPC G16H 30/20; A61B 8/465; A61B 8/54; G06F 3/0482

(57) ABSTRACT

Systems are provided for providing context-based chatbot assistance during an ultrasound imaging workflow. In one example, an ultrasound imaging system includes an artificial intelligence (AI) processing circuit. The AI processing circuit is configured to receive image data obtained by a transducer; generate a natural language input based on the image data configured to prompt a response; input the natural language input to a machine learning model during an ultrasound imaging workflow performed by the ultrasound imaging system; identify, from a database of contextual information relating to the ultrasound imaging system, a portion of the contextual information regarding the ultrasound imaging workflow; generate the response based on the portion of the contextual information and the processed image data; and provide the response.

### FIELD

Embodiments of the subject matter disclosed herein relate to ultrasound imaging, and more particularly, to providing assistance during an ultrasound imaging workflow using contextual information regarding the ultrasound imaging workflow.

### BACKGROUND

During a medical imaging workflow, a plurality of medical images of a patient are obtained by a technician, such as a sonographer, to measure or detect various aspects of anatomical features present within the medical images. These images and measurements are subsequently analyzed by a clinician, such as a cardiologist or radiologist, to observe a condition or to identify any abnormalities.

### SUMMARY

An embodiment relates to an ultrasound imaging system. The ultrasound imaging system includes a transducer configured to transmit and receive an ultrasound signal, a matching layer configured to have an acoustic impedance between a tissue to be imaged and a material of the transducer, a damping block configured to absorb ultrasound energy, and an artificial intelligence (AI) processing circuit. The AI processing circuit is configured to process image data obtained by the transducer. The AI processing circuit is configured generate a natural language input configured to prompt a response from the AI processing circuit based on the natural language input. The AI processing circuit is configured to input the natural language input to a machine learning model during an ultrasound imaging workflow performed by the ultrasound imaging system. The AI processing circuit is configured to identify, from a database of contextual information relating to the ultrasound imaging system, a portion of the contextual information regarding the ultrasound imaging workflow. The AI processing circuit is configured to generate the response based on the portion of the contextual information and the processed image data. The AI processing circuit is configured to provide, to a user via a display of the ultrasound imaging system, the response.

Another embodiment relates to an ultrasound imaging system. The ultrasound imaging system includes a transducer configured to transmit and receive an ultrasound signal, a matching layer configured to have an acoustic impedance between a tissue to be imaged and a material of the transducer, a damping block configured to absorb ultrasound energy, and a processing circuit. The processing circuit includes a processor coupled to a memory device, and the memory device stores instructions thereon that, when executed, cause the processing circuit to perform operations including processing, using an artificial intelligence (AI) processing circuit, image data obtained by the transducer, generating a natural language input configured to prompt a response from the AI processing circuit based on the natural language input, inputting the natural language input to a machine learning model during an ultrasound imaging workflow performed by the ultrasound imaging system, identifying, from a database of contextual information relating to the ultrasound imaging system, a portion of the contextual information related to the ultrasound imaging workflow, generating, using the AI processing circuit, the response based on the portion of the contextual information and the processed image data, and providing, to a user via a display of the ultrasound imaging system, the response.

Another embodiment relates to a method. The method includes processing, by an artificial intelligence (AI) processing circuit, image data obtained by an ultrasound probe of an ultrasound imaging system. The method includes generating, by the AI processing circuit, a natural language input configured to prompt a response from the AI processing circuit based on the natural language input. The method includes inputting, by the AI processing circuit, the natural language input to a machine learning model during an ultrasound imaging workflow performed by the ultrasound imaging system. The method includes identifying, by the AI processing circuit and from a database of contextual information relating to the ultrasound imaging system, a portion of the contextual information regarding the ultrasound imaging workflow. The method includes generating, by the AI processing circuit, the response based on the portion of the contextual information and the processed image data. The method includes providing, by the AI processing circuit and to a user via a display, the response.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a block diagram of an ultrasound imaging system, according to an example embodiment.

FIG. 2 is an illustration of the ultrasound imaging system of FIG. 1, according to an example embodiment.

FIG. 3 is a block diagram of an artificial intelligence (AI) circuit used in the ultrasound imaging system of FIG. 1, according to an example embodiment.

FIG. 4A is a block diagram of a process for providing chatbot assistance using the ultrasound imaging system of FIG. 1, according to an example embodiment.

FIG. 4B is a block diagram of the process of FIG. 4A for providing chatbot assistance using the AI circuit of FIG. 3, according to an example embodiment.

FIG. 5 is a flow chart illustrating a method for providing context-based assistance during an ultrasound imaging workflow using the ultrasound imaging system of FIG. 1, according to an example embodiment.

FIG. 6 is a flow chart illustrating a method for generating pre-defined prompts during the method of FIG. 5, according to an example embodiment.

FIG. 7 is an illustration of a user interface with chatbot assistance, according to an example embodiment.

FIG. 8 is an illustration of a process for generating and providing the chatbot response during the method of FIG. 5 using the interface of FIG. 7, according to an example embodiment.

FIG. 9 is an illustration of an ultrasound image including a user input, according to an example embodiment.

FIG. 10A is a textual illustration of contextual information used to provide chatbot assistance during the method of FIG. 5, according to an example embodiment.

FIG. 10B is a graphical illustration of the contextual information of FIG. 10A, according to an example embodiment.

### DETAILED DESCRIPTION

Referring generally to the figures, systems and methods for providing context-based assistance during an ultrasound imaging workflow are disclosed. The systems and methods disclosed herein use artificial intelligence (AI) to process image data and contextual information to identify data/information relating to an input from a user performing the ultrasound imaging workflow.

The implementations described herein address a technical problem by providing enhanced data integration and analysis capabilities, which deliver a particular technical solution that streamlines and refines ultrasound imaging workflows. The systems described herein are implemented to improve how data is synthesized and utilized from various sources that provide information relating to an ultrasound imaging workflow. By integrating data related to a specific procedure, technician, patient, and so on, these systems provide real-time, chatbot assistance based on a current status of the ultrasound imaging workflow. That is, with the context-based approach described herein, the chatbot assistance corresponds to an exact situation that a sonographer is in while interacting with the chatbot. For example, the implementations can provide assistance to a sonographer based on a user manual associated with an ultrasound imaging system being used. In another example, the implementations can provide assistance to a sonographer based on medical literature relating to a particular anatomical region being captured during the ultrasound imaging procedure. Accordingly, this approach provides a specific technical improvement to various technical problems, including those set forth herein.

The systems described herein may also reduce processing power by performing various processing operations simultaneously to provide chatbot assistance in real-time during the ultrasound imaging workflow, rather than performing a plurality of processing operations individually and consuming unnecessary processing power. Furthermore, the systems as described herein generate chatbot assistance configured to assist a sonographer in executing a complete ultrasound scan given various pieces of contextual information (e.g., industry standards, sonographer preferences, patient medical history, etc.). That is, the systems as described herein are trained to identify imaging parameters and operations required to obtain a complete scan in a given ultrasound procedure, therefore ensuring that the scan is complete prior to attempting to process the ultrasound images. This consideration of contextual information when providing chatbot assistance during the ultrasound imaging workflow reduces processing power by avoiding collection of unnecessary ultrasound data and submission of an incomplete scan, which can cause the sonographer to have to capture additional images during a successive scan. Additionally, providing pre-configured prompts (e.g., buttons) for submitting an input to the chatbot (e.g., “Help me with my exam,” “What is missing in my exam,” etc.) reduces processing power and improves bandwidth because a natural language input is pre-encoded into the prompts such that a user can select a button with the desired natural language input rather than submitting the desired natural language input as a textual input or a voice input.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

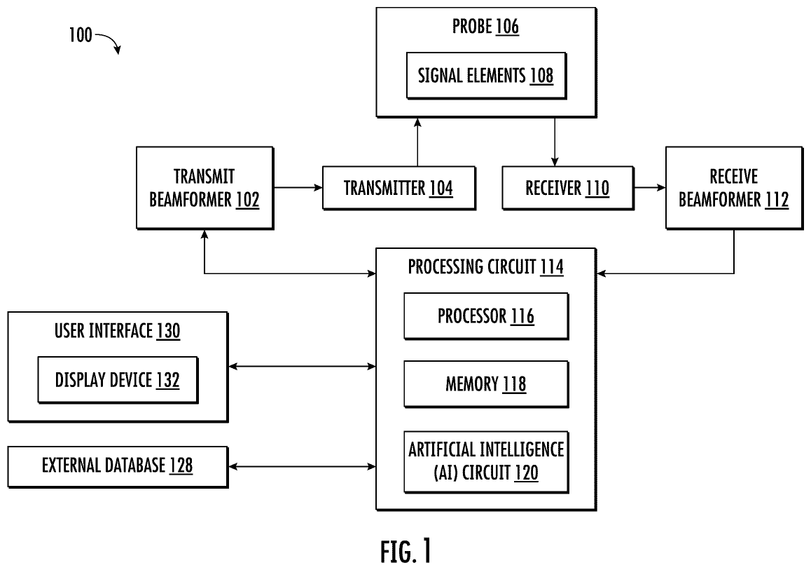

Referring to FIG. 1, a schematic diagram of an ultrasound imaging system 100 is shown. The ultrasound imaging system 100 may be used in a medical environment (e.g., hospitals, clinics, etc.), for example, by a sonographer, technician, or other clinician certified to collect ultrasound data from a patient.

An example of a procedure performed using the ultrasound imaging system 100 may be an echocardiogram. Echocardiograms are performed to detect heart abnormalities in a patient by collecting and processing ultrasound data (e.g., using the ultrasound imaging system 100, as described herein). During an echocardiogram, a sonographer follows a particular imaging protocol specific to echocardiography. The echocardiography-specific imaging protocol ensures that the heart is thoroughly captured by the ultrasound data and that the processing of the ultrasound data is focused on detecting heart abnormalities. The sonographer collects the ultrasound data by navigating a probe (e.g., probe 106, as described below) over the patient's chest until a sufficient volume of ultrasound images are collected. The collected images are stored in a central storage device (e.g., memory 118) and analyzed by the sonographer. The sonographer generates a set of measurements from the images (e.g., 50-100 records), and the images and measurements are collectively reviewed by a cardiologist. The cardiologist provides any clinical findings/conclusions in a report submitted to the patient's medical record.

As shown in FIG. 1, the ultrasound imaging system 100 includes a transmit beamformer 102, a transmitter 104, a probe 106, a receiver 110, and a receive beamformer 112.

The transmit beamformer 102 may be either a hardware beamformer or a software beamformer. In embodiments where the transmit beamformer 102 is a hardware beamformer, the transmit beamformer 102 may include one or more of a graphics processing unit (GPU), a microprocessor, a central processing unit (CPU), a digital signal processor (DSP), or any other type of processor capable of performing logical operations. The transmit beamformer 102 may be configured to perform conventional beamforming techniques as well as techniques such as retrospective transmit beamforming (RTB). Alternatively, in embodiments where the transmit beamformer 102 is a software beamformer, a processor (e.g., processor 116, as described below) may be configured to perform some or all of the functions associated with the transmit beamformer 102.

The probe 106 may be a linear array probe, a curvilinear array probe, a sector probe, or any other type of probe configured to obtain two-dimensional (2D) B-mode data and 2D color flow data. Alternatively or additionally, the probe 106 may be any type of probe configured to obtain 2D B-mode data and data corresponding to another ultrasound mode that detects blood flow velocity in the direction of a vessel axis. In some embodiments, the probe 106 may include a position sensor configured to detect a position of the probe 106 relative to one or more reference locations. That is, the position sensor may continuously track movement (e.g., rotation, translation, orientation, etc.) of the probe 106 relative to the location of the probe 106 when the anatomy being imaged is identified. For example, the anatomy being imaged may be identified as a left atrial appendage (LAA) at a first location of the probe 106. Then, the position sensor may track the movement of the probe 106 relative to the LAA in order to identify successive locations of the probe 106. In some embodiments, the position sensor may transmit position data to be stored within the ultrasound imaging system 100 (e.g., in memory 118).

The probe 106 may include a transducer configured to transmit and receive an ultrasound signal. In some embodiments, as shown in FIG. 1, the probe 106 includes signal elements 108. The signal elements 108 may be arranged in a transducer array, and in some embodiments may be arranged in a one-dimensional (1D) or 2D array. The transmit beamformer 102 and the transmitter 104 drive the signal elements 108 to emit pulsed ultrasonic signals into a body of a subject (e.g., a patient). For example, during an echocardiogram, a sonographer or other clinician may navigate the probe 106 over a patient's chest so that the signal elements 108 in the probe 106 emit the pulsed ultrasonic signals into the patient's thoracic cavity. The pulsed ultrasonic signals are then back-scattered from anatomical structures in the body, such as blood cells or muscular tissues, to produce echoes that return to the signal elements 108. That is, the signal elements 108 may include the transducer configured to transmit and receive the ultrasound signal, a matching layer configured to have an acoustic impedance between a tissue to be imaged and a material of the transducer (e.g., such that the pulsed electronic signals can be back-scattered from the anatomical structures in the body and received as echoes by the signal elements 108), and a damping block configured to absorb ultrasound energy.

The receiver 110 receives the echoes from the probe 106 and converts the echoes into electrical signals. The electrical signals are then passed through the receive beamformer 112, which produces the ultrasound data from the electrical signals. As described above with reference to the transmit beamformer 102, the receive beamformer 112 may be either a hardware beamformer or a software beamformer. In embodiments where the receive beamformer 112 is a hardware beamformer, the receive beamformer 112 may include one or more of a GPU, a microprocessor, a CPU, a DSP, or any other type of processor capable of performing logical operations. The receive beamformer 112 may be configured to perform conventional beamforming techniques as well as techniques such as retrospective transmit beamforming (RTB). Alternatively, in embodiments where the receive beamformer 112 is a software beamformer, a processor (e.g., processor 116, as described below) may be configured to perform some or all of the functions associated with the receive beamformer 112.

Although the transmit beamformer 102, the transmitter 104, the receiver 110, and the receive beamformer 112 are shown in FIG. 1 as being components of the ultrasound imaging system 100 that are distinct from the probe 106, it should be appreciated that in some embodiments, the probe 106 may include electronic circuitry configured to perform the functions of each of the transmit beamformer 102, the transmitter 104, the receiver 110, and/or the receive beamformer 112. That is, all or part of the transmit beamformer 102, the transmitter 104, the receiver 110, and/or the receive beamformer 112 may be situated within the probe 106.

Referring still to FIG. 1, the ultrasound imaging system 100 is shown to include a processing circuit 114. As shown, the processing circuit 114 may include at least one processor 116, a memory 118, and an artificial intelligence (AI) circuit 120. In this way, the processing circuit 114 may be structured or configured to execute or implement the instructions, commands, and/or control processes described herein with respect to the processor 116, the memory 118, and the AI circuit 120. While shown as being separate from the probe 106 in FIG. 1, it will be appreciated that the processing circuit 114 can be part of the probe 106. For example, the processing circuit 114 can be disposed in a handheld housing of the probe 106 (e.g., in the case of the probe 106 being a wireless probe).

The processor 116 may include a CPU, a GPU, a microprocessor, a DSP, a general-purpose single- or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 116 may be shared by multiple circuits (e.g., the circuits of the processor 116 may include or otherwise share the same processor which, in some example embodiments, may execute instructions stored, or otherwise accessed, via different areas of the memory 118). Alternatively or additionally, the processor 116 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

The processor 116 may be configured to control the transmit beamformer 102, the transmitter 104, the receiver 110, and the receive beamformer 112. The processor 116 may also be in electronic communication with the probe 106. For purposes of this disclosure, the term “electronic communication” may be defined to include both wired and wireless communications.

In some embodiments, the processor 116 may be configured to control the probe 106 during data acquisition. That is, the processor 116 may control the data acquisition by controlling which of the signal elements 108 are active and by controlling a shape of the beam emitted from the probe 106. Alternatively or additionally, the processor 116 may include a complex demodulator configured to demodulate radio frequency (RF) data obtained by the probe 106 and generate raw data. According to other embodiments, the demodulation of the RF data may be performed by another component of the ultrasound imaging system 100. The processor 116 may perform the processing operations described herein according to a plurality of selectable ultrasound modalities.

Depending on a mode of operation of the ultrasound imaging system 100, the processor 116 may process ultrasound data obtained by the probe 106 according to the mode of operation to generate 2D or 3D image data. For example, the mode of operation may include B-mode, color flow Doppler mode, M-mode, color M-mode, spectral Doppler, elastography, TVI, strain, strain rate, and the like. Various of these modes of operation may be configured to, for instance, convert ultrasound data from beam space coordinates (e.g., received from the receive beamformer 112) to display space coordinates (e.g., such that the ultrasound data may be displayed as image data). In some embodiments, the mode of operation may allow for video processing by the processor 116 such that a series of images (e.g., processed ultrasound data) may be displayed in real-time while a scanning session/procedure is being performed on a patient. An operator of the ultrasound imaging system 100 (e.g., a sonographer) may switch between various modes in order to obtain a variety of ultrasound data and to perform a complete scan of an anatomical region of interest. For example, as described herein, the operator may switch between modes using user interface 130 (e.g., using physical controls, interface inputs representing physical controls, etc.).

The processor 116 performs the processing operations in real-time as the echo signals are received by the receiver 110 from the probe 106. For the purposes of this disclosure, the term “real-time” is defined to include a procedure that is performed without any intentional delay. As an illustrative, non-limiting example, in certain instances, the ultrasound imaging system 100 may obtain images at a real-time volume-rate of 7-20 volumes/sec. It should be appreciated, however, that the real-time volume-rate may be dependent on the length of time that it takes to obtain each volume of data for display. Thus, the ultrasound imaging system 100 may be configured to obtain 2D data of an anatomical region at a faster rate than 3D data of the same anatomical region because it takes longer to obtain a volume of 3D data than the same volume of 2D data. Similarly, when the ultrasound imaging system 100 obtains a relatively large volume of data, the real-time volume-rate may be slower than for a smaller volume of data. For example, during an abdominal scan, the real-time volume-rate may be slower if the patient is an adult versus if the patient is an infant because the volume of data is larger for the adult than for the infant (e.g., due to the abdomen of an adult being larger than the abdomen of an infant). Therefore, certain implementations of the ultrasound imaging system 100 may have real-time volume-rates that are faster than 20 volumes/sec, while other implementations of the ultrasound imaging system 100 may have real-time volume-rates that are slower than 7 volumes/sec.

In some embodiments, the ultrasound imaging system 100 may include multiple processors configured to perform the processing operations/functionality described with reference to processor 116. For example, in such embodiments, a first processor of the multiple processors may be configured to demodulate and decimate the RF signal while a second processor of the multiple processors may be configured to further process the RF data prior to displaying an image representative of the data. It should be appreciated that other embodiments may use a different arrangement of processors.

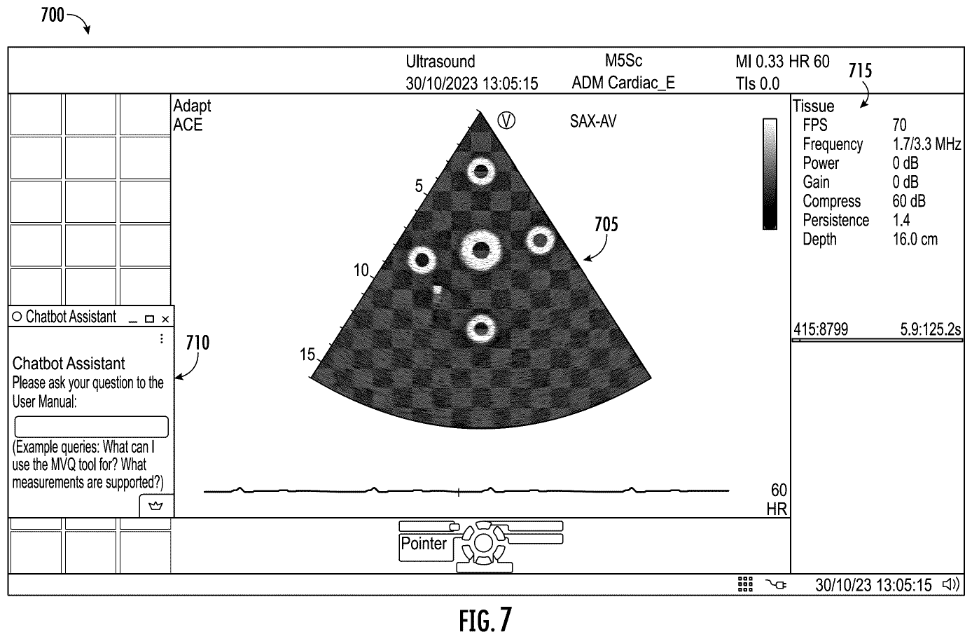

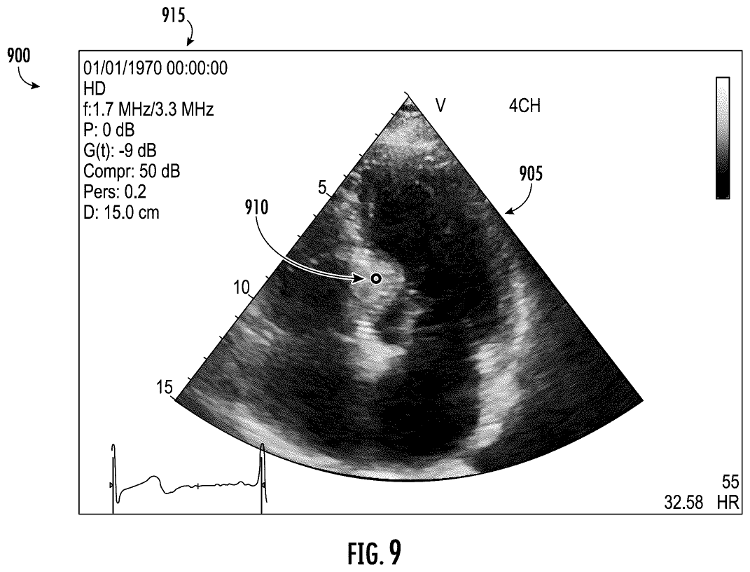

The processor 116 may also be in electronic communication with the display device 132 such that the processor 116 may process ultrasound data obtained by the probe 106 and generate images to display on the display device 132 (e.g., ultrasound image 705 and ultrasound image 905, as described below with reference to FIGS. 7 and 9, respectively).

As shown in FIG. 1, the processing circuit 114 also includes the memory 118. The memory 118 may be configured to, for example, store processed volumes of data obtained by the ultrasound imaging system 100 (e.g., ultrasound data collected by the probe 106). For example, the memory 118 may be a hospital picture archiving and communication system (PACS). The memory 118 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 118 may be or include tangible, non-transient volatile memory or non-volatile memory. The memory 118 may also include database components, object code components, script components, or any other type of information structure for supporting the activities and information structures described in the present application.

In various embodiments, the memory 118 may have varying capacity (e.g., storage space) across embodiments of the ultrasound imaging system 100. For example, the memory 118 may be configured to store at least 60 minutes' worth of ultrasound data. The ultrasound data may be stored in the memory 118 such that the ultrasound data may be retrieved according to an order/time of acquiring the data. That is, the ultrasound data may be stored with a timestamp indicating a time at which the ultrasound data was collected and may be retrieved starting with an oldest time at which the ultrasound data was collected.

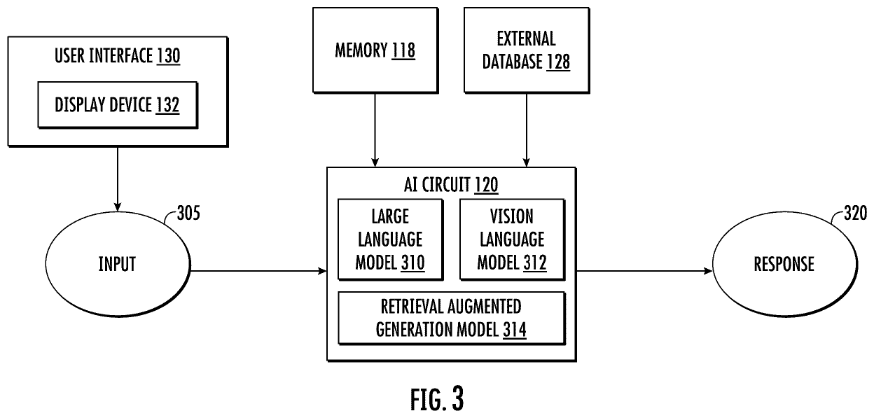

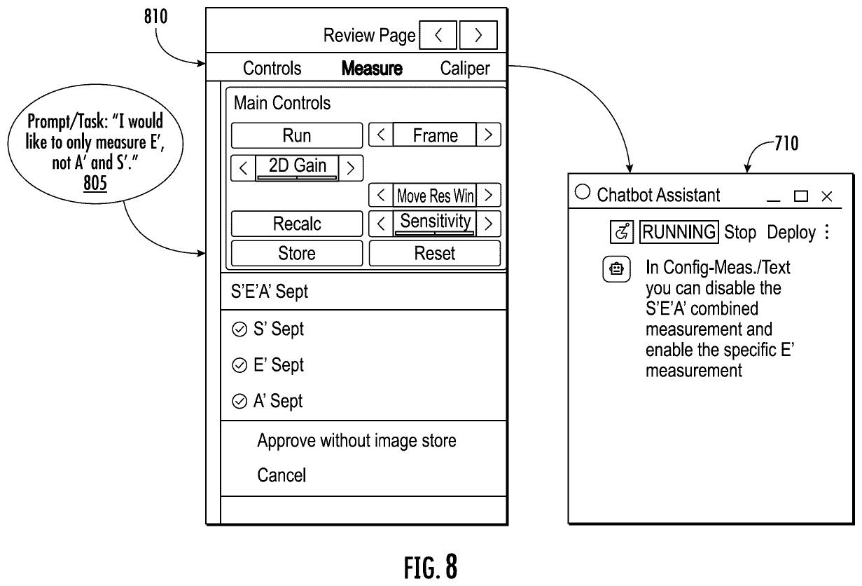

The processing circuit 114 also includes the AI circuit 120. As described herein, the AI circuit 120 may be configured to perform various operations relating to providing context-based chatbot assistance during an ultrasound imaging workflow. In this way, the AI circuit 120 may be configured to provide a chatbot interface (e.g., the chatbot assistance window 710, as described below with reference to FIGS. 7-8) via the display device 132. The various operations performed by the AI circuit 120 are described in greater detail below, with reference to FIGS. 3-10B.

The ultrasound imaging system 100 may also include an external database 128 and a user interface 130. The external database 128 refers to a database from which the processing circuit 114 (e.g., the AI circuit 120) retrieves information used in providing intelligent assistance during an ultrasound imaging workflow. For example, the external database 128 may be a medical information database. The medical information database may store clinical guidelines, standard practices, medical literature, medical textbooks, published research, previous case studies, and so on. Depending on an implementation of the ultrasound imaging system 100 and/or a procedure performed thereby, the AI circuit 120 may retrieve clinical guidelines, standard practices, medical literature, medical textbooks, published research, and previous case studies related to the implementation and/or procedure. For example, if the ultrasound imaging system 100 is being used in a hospital setting to perform an LAA closure procedure, the AI circuit 120 may retrieve clinical guidelines and standard practices related to the hospital setting and the LAA closure procedure. Continuing with this example, the AI circuit 120 may also retrieve information from the medical literature, medical textbooks, published research, and previous case studies related to cardiac anatomy and the LAA closure procedure.

The user interface 130 may be used by a sonographer or other clinician to control operation of the ultrasound imaging system 100. For example, the sonographer may use the user interface 130 to control the input of patient data, to change a scanning or display parameter, and/or to select various other modes, operations, parameters, etc. of the ultrasound imaging system 100. Furthermore, as described herein, the sonographer may use the user interface 130 to interact with a chatbot configured to provide assistance to the sonographer throughout an ultrasound imaging workflow. For example, the sonographer may submit a question or other prompt to the chatbot using the user interface 130 (e.g., by typing a text entry, by speaking into a microphone, by selecting a selectable element on a touch screen, etc.). The sonographer may also receive a response from the chatbot via the user interface 130 (e.g., as a textual output presented on display device 132, as an audible output via a speaker, etc.).

In some embodiments, the user interface 130 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. Alternatively, in other embodiments, the user interface 130 may be an electronic device that was designed and developed for use in a medical environment.

According to some embodiments, the user interface 130 may be physically separate from the rest of the ultrasound imaging system 100 (e.g., the transmit beamformer 102, the transmitter 104, the probe 106, the receiver 110, the receive beamformer 112, the processing circuit 114, and/or the external database 128). The user interface 130 may communicate with the processor 116 through a wireless protocol, such as Wi-Fi, Bluetooth, wireless local area network (WLAN), near-field communication, and so on. According to some embodiments, the user interface 130 may communicate with the processor 116 through an application programming interface (API).

In some embodiments, the user interface 130 may include physical controls such as one or more of buttons, sliders, a rotary knob, a mouse, a keyboard, a trackball, hard keys linked to specific actions, soft keys that may be configured to control different functions, and so on. The user interface 130 may also include a microphone configured to receive audio inputs (e.g., speech inputs) from a user and a speaker configured to provide audio outputs to the user. In some instances, the ultrasound imaging system 100 may be equipped to perform voice recognition of the audio inputs received from the user interface 130 such that the ultrasound imaging system 100 may identify when an operator of the ultrasound imaging system 100 (e.g., a sonographer) is speaking versus when a patient and/or other personnel are speaking.

As shown in FIG. 1, the user interface 130 may also include a display device 132. In some embodiments, the display device 132 may be configured to display a graphical user interface (GUI) based on an instruction from the memory 118. The GUI may include user interface icons representing commands and instructions relating to the operation of the ultrasound imaging system 100. The user interface icons of the GUI may be configured such that a user (e.g., the sonographer, clinician, etc.) may select a specific user interface icon in order to initiate a specific function controlled by the GUI. For example, various user interface icons may be used to represent windows, menus, buttons, cursors, scroll bars, and so on. That is, the physical controls of the user interface 130 may be included as individual hardware elements, as user interface icons displayed on the display device 132, or as a combination of hardware elements and user interface icons.

In some embodiments, the display device 132 may include a touch-sensitive display device or a touch screen. According to such embodiments, the touch screen may be configured to interact with the GUI displayed by the display device 132 such that a user (e.g., the sonographer) can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user. In some embodiments, the touch screen may be incorporated as part of the display device 132 or may be separate from the display device 132. The user interface 130 may also include a proximity sensor configured to detect objects and/or gestures that are within a predetermined distance (e.g., five feet, six inches, ten centimeters, etc.) of the proximity sensor. In various embodiments, the proximity sensor may be located on the display device 132 or as part of a touch screen that is separate from the display device 132.

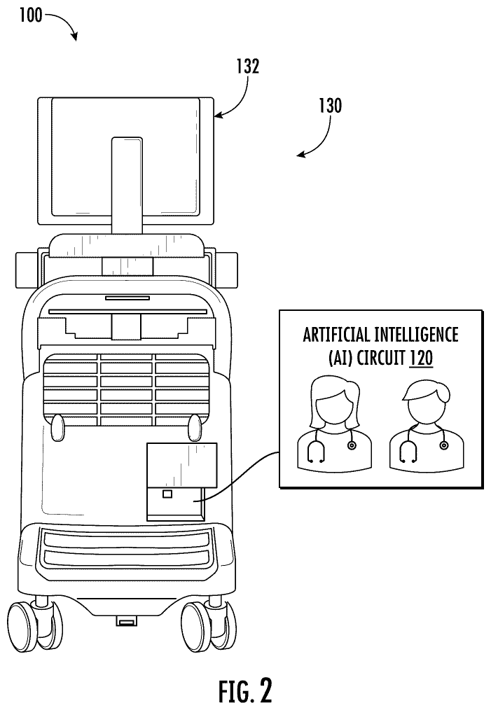

Referring to FIG. 2, an illustration of the ultrasound imaging system 100 is shown. As shown, FIG. 2 depicts the user interface 130 and the display device 132. Additionally, a schematic of the AI circuit 120 is shown. More specifically, the schematic of the AI circuit 120 includes depictions of medical personnel (e.g., a doctor, a surgeon, a nurse, a technician, a clinician, etc.). That is, the medical personnel depicted within the AI circuit 120 of FIG. 2 represent the medical expertise provided to a user of the ultrasound system 100 by the chatbot assistance described herein. In this way, the user of the ultrasound imaging system 100 may interact with the chatbot (e.g., represented by the medical personnel depicted within the AI circuit 120) during an ultrasound imaging workflow using the display device 132 of the user interface 130 in a same way as the user of the ultrasound imaging system 100 may interact with live medical personnel (e.g., to ask questions, receive instructions/guidance, etc.) during the ultrasound imaging workflow.

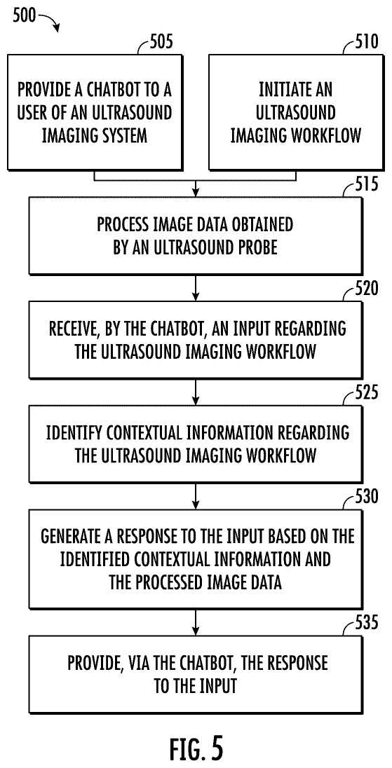

Referring to FIG. 3, the AI circuit 120 of the ultrasound imaging system 100 is shown in greater detail. The AI circuit 120 is shown to receive input 305 from the display device 132 of the user interface 130. In some embodiments, the input 305 refers to the input received at step 520 of method 500, as described in greater detail below with reference to FIG. 5.

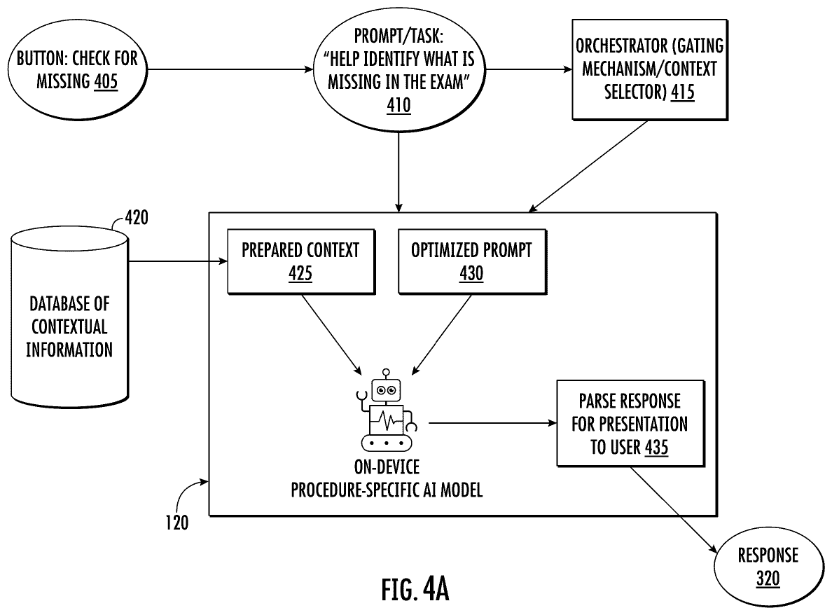

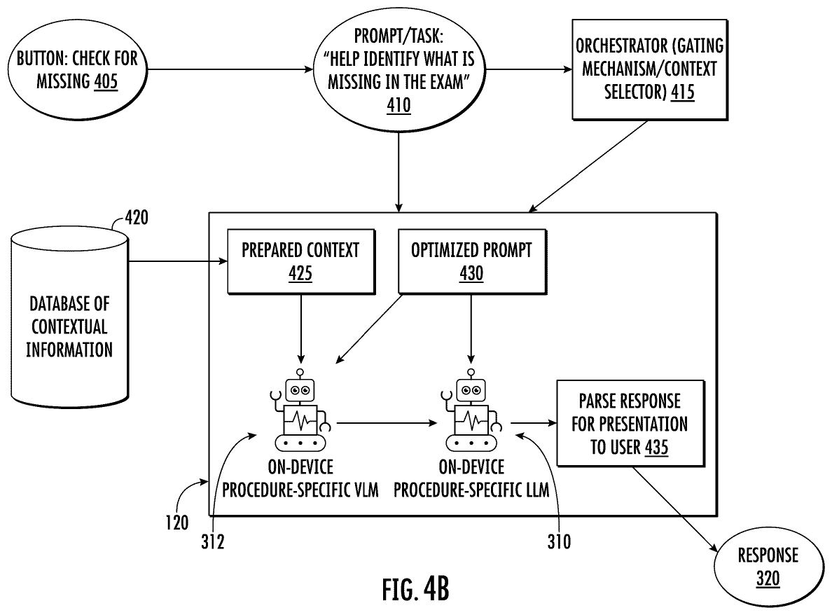

As shown in FIG. 3, the AI circuit 120 may include at least one of a large language model (LLM) 310 or a vision language model (VLM) 312. The LLM 310 is configured to receive natural language (e.g., text) inputs, while the VLM 312 is configured to receive graphical (e.g., visual) inputs. For instance, a textual query (e.g., received via a free-text box of the chatbot assistance window 710, received via selection of a pre-configured button 405, etc.) may be received and processed using the LLM 310, while the image data captured by the probe 106 may be received and processed using the VLM 312. In some embodiments, as shown here and in FIG. 4B, the AI circuit 120 includes the LLM 310 and the VLM 312.

The AI circuit 120 may be configured to access the memory 118 such that information contained therein (e.g., a stored collection of ultrasound images, a user manual/guide associated with the ultrasound imaging system 100, patient medical history, medical research data, etc.) may be retrieved as the information relates to the input 305. For example, if the input 305 includes a selection of a “What is missing from my exam” button (e.g., button 405, as described below with reference to FIGS. 4A-4B), the AI circuit 120 may access the stored collection of ultrasound images from the memory 118 that have been collected thus far during the ultrasound imaging workflow to identify whether any necessary images are still missing. As another example, if the results of the ultrasound imaging workflow are being used in a research initiative, the research initiative may require specific ultrasound data to be collected, and such instructions relating to participation in the research initiative may be retrieved by the AI circuit 120 from the memory 118.

In some embodiments, the AI circuit 120 may be powered by a retrieval augmented generation (RAG) model 314. The RAG model 314 may be configured to access the external database 128 such that the information contained therein (e.g., clinical guidelines, standard practices, medical literature, medical textbooks, published research, or previous case studies) may be retrieved as the information relates to the input 305. With the RAG model 314, the AI circuit 120 may be configured to generate the response 320 to the input 305 using information relating to a specific procedure (e.g., an echocardiogram), a specific setting (e.g., a hospital), a specific anatomy (e.g., a heart), and so on, without requiring a retraining of the LLM 310 and/or the VLM 312 for uses relating to each of the specific procedure, the specific setting, the specific anatomy, and so on.

The information relating to the input 305 retrieved from the memory 118 and from the external database 128 may then be combined with the input 305 to create an augmented input. That is, the input 305 is augmented with the relevant information retrieved from the memory 118 and the external database 128. The augmented input may be applied as an input to the LLM 310 and/or the VLM 312 such that the response 320 from the AI circuit 120 is based on the augmented input. The response 320 may refer to the response generated at step 530 of method 500, as described below with reference to FIG. 5.

Referring to FIGS. 4A and 4B, a block diagram of a process for providing chatbot assistance using the ultrasound imaging system 100 (e.g., the AI circuit 120) is shown. The process may begin when a user of the ultrasound imaging system 100 submits an input to the chatbot. As shown, the input may be submitted by interacting/engaging with a “Check for Missing” button 405. The button 405 may be configured to automatically, once engaged with by a user, submit an input to the chatbot requesting guidance regarding the ultrasound imaging workflow. That is, the button 405 may be encoded with a natural language input configured to prompt a response from the chatbot. More specifically, and as shown in FIGS. 4A and 4B, the “Check for Missing” button 405 may be encoded, by the ultrasound imaging system 100, with the natural language prompt/task 410: “Help identify what is missing in the exam. ” In other words, the chatbot is instructed to identify whether any images, steps, data, and so on are missing from the images, steps, data, and so on that have been collected/performed thus far in the ultrasound imaging workflow. In this instance, the ultrasound imaging system 100 generates the natural language input rather than the user (e.g., as in a conventional chatbot text conversation), and the user submits the pre-generated natural language input to the chatbot by engaging with a button (e.g., button 405) included on an interface displayed by the ultrasound imaging system 100.

In some embodiments, and as shown, the prompt/task 410 may be provided to an orchestrator 415. The orchestrator 415 may refer to a gating mechanism configured to identify relevant context relating to the prompt/task 410. In some embodiments, the orchestrator 415 may be configured to process the context of the prompt/task 410 to identify a type of response that is expected by the user in response to the user input (e.g., selection of the button 405). The type of response may be an answer to a question, an initiation of an action relating to the ultrasound imaging system 100, etc. An output from the orchestrator 415 may be provided to the AI circuit 120 such that the chatbot may provide an appropriate response 320 based on a type of expected response identified by the orchestrator 415.

The prompt/task 410 may also be provided to the AI circuit 120. As shown the AI circuit 120 is configured to, based on the received prompt/task 410 and the output from the orchestrator 415, generate a prepared context 425 and an optimized prompt 430. The AI circuit 120 may generate a prepared context 425 using a database of contextual information 420. The database of contextual information 420 refers to a collection of data/information accessible by the ultrasound imaging system 100. In this way, the database of contextual information 420 may include internal information (e.g., stored in the memory 118) and/or external information (e.g., retrieved from the eternal database 128). In some embodiments, the internal information may include patient medical history and/or a user guide associated with the ultrasound imaging system 100. The external information may refer to information from the medical information database, as described above with reference to FIG. 1. Further, the database of contextual information 420 may include real-time information regarding an ultrasound imaging workflow using the ultrasound imaging system 100. That is, in some embodiments, the real-time information may include a real-time status of the ultrasound imaging workflow and/or a current configuration of the display of the ultrasound imaging system.

The prepared context 425 using the database of contextual information 420 and the optimized prompt 430 may be provided to an on-device (e.g., located within the ultrasound imaging system 100) procedure-specific (e.g., cardiac-specific, abdominal-specific, prenatal-specific, etc.) AI model (e.g., illustrated as a chatbot). The on-device procedure-specific AI model outputs a response, which is parsed at block 435 for presentation to a user as the response 320, (e.g., the response provided at step 535 of method 500).

Referring to FIG. 4B, the AI circuit 120 is shown to include an on-device procedure-specific VLM (e.g., the VLM 312) and an on-device procedure-specific LLM (e.g., the LLM 310). In such embodiments, the prepared context 425 and the optimized prompt may be received by the VLM 312. Then, an output from the VLM 312 based on the prepared context 425 and the optimized prompt 430 may be received by the LLM 310, along with the optimized prompt 430, such that the LLM 310 outputs a response. The response from the LLM 310 is parsed at block 435 for presentation to the user as response 320.

Referring to FIG. 5, a flow chart is shown illustrating a method 500 for providing context-based chatbot assistance during an ultrasound imaging workflow using an ultrasound imaging system. In at least one embodiment, the ultrasound imaging system referred to by method 500 is the ultrasound imaging system 100 described above with reference to FIGS. 1-4B, and method 500 may be implemented by the ultrasound imaging system 100. In some embodiments, method 500 may be implemented as executable instructions in a memory of the ultrasound imaging system 100, such as the memory 118 of FIG. 1.

Prior to initiating a collection of ultrasound data, method 500 may begin when an operator (e.g., a sonographer, technician, or other clinician) is authenticated as an authorized user of the ultrasound imaging system 100. In some embodiments, the operator may authenticate themselves as an authorized user of the ultrasound imaging system 100 by logging in to a portal (e.g., an online application accessible via the user interface 130) associated with the environment in which the ultrasound imaging system 100 is being implemented (e.g., a hospital or other healthcare provider). For instance, the operator may log in using a unique identifier (e.g., a username, a password, a biometric scan, a pin code, etc.).

After the operator is authenticated, the operator of the ultrasound imaging system 100 may enter patient and/or procedure-specific information into the ultrasound imaging system 100 prior to the collection of ultrasound data. For example, the operator may submit patient information (e.g., identifying information such as name, date of birth, social security number, and so on, and/or medical information such as a medical history, family medical history, a current diagnosis, and so on) via the user interface 130. In some embodiments, the operator may select the patient from a list of patients (e.g., patients associated with a scheduled procedure to be performed by the operator), and the patient information may be imported to the ultrasound imaging system 100 (e.g., from a database associated with the environment in which the ultrasound imaging system 100 is being implemented, such as a hospital).

In addition to the patient information, the operator may enter (e.g., as a text entry) or otherwise select (e.g., from a drop-down list of procedures) the procedure that the operator is preparing to perform. For example, the operator may enter or select “echocardiogram” as the procedure. Additionally, the operator may enter or otherwise select any known pathologies or other medical conditions that may be relevant to the procedure. For instance, the operator may be performing an ultrasound examination on the patient in preparation for an LAA closure procedure, and such information may be entered into the ultrasound imaging system 100 prior to the collection of ultrasound data. In this way, the ultrasound imaging system 100 may be configured to prepare intelligent guidance regarding the ultrasound imaging workflow, as described herein, specific to data that may be relevant to the LAA closure procedure (e.g., sufficient imaging of the patient's heart and, more specifically, the left atrium).

Additionally, prior to initiating the collection of ultrasound data, method 500 may include the operator of the ultrasound imaging system 100 selecting an operating mode of the ultrasound imaging system 100. In some embodiments, the operating mode may refer to an imaging mode of an ultrasound probe (e.g., probe 106). For example, the operating mode may include any of the imaging modes described above, such as the B-mode, color flow Doppler mode, M-mode, Color M-mode, spectral Doppler, Elastography, TVI, strain, strain rate, and the like. In some embodiments, the operator may select the imaging mode of the probe 106 via a user device (e.g., the user interface 130). For example, the operator may select the imaging mode via a GUI presented on a touch screen display device (e.g., display device 132). Alternatively or additionally, the operator may engage with a button or other physical control located on the probe 106 and configured to control the operating mode. Each operating mode may correspond to a particular method of operation of the probe 106. For example, the operating mode may be configured to control signals transmitted to the signal elements 108 of the probe 106 and/or process signals received from the signal elements 108 of the probe 106 according to a particular method.

As shown in FIG. 5, at step 505, method 500 may include providing a chatbot to a user of the ultrasound imaging system 100. In some instances, the chatbot may be provided at step 505 by the AI circuit 120 as an interface element displayed on the display device 132. For example, providing the chatbot at step 505 may include providing the chatbot assistance window 710 to a user via the GUI 700, as described below with reference to FIGS. 7-8.

In some embodiments, the chatbot is provided at step 505 when an operator of the ultrasound imaging system 100 initiates an ultrasound imaging workflow at step 510. That is, initiating the ultrasound imaging workflow at step 510 may include beginning a collection of ultrasound data using the probe 106. The ultrasound data may refer to a set of ultrasound images depicting a patient's anatomy (e.g., including ultrasound image 905, as described below). After the ultrasound data is collected by the probe 106 and converted to image data by the processing circuit 114 (e.g., by the processor 116, as described above), the images may be stored in the memory 118. The image data may include individual images and/or a cine loop (e.g., a series of five images, ten images, twenty images, etc.). The cine loop refers to a series of images relating to an anatomical region captured sequentially by the probe 106. In some embodiments, additional image data may be stored continuously in the memory 118 as a scanning session progresses and new ultrasound data is obtained. Where the memory 118 has a limited capacity (e.g., storage for 100 images), in some embodiments, older images in the set of ultrasound images may be replaced by new images as new ultrasound data is obtained by the probe 106 and stored in the memory 118.

At step 515, image data obtained by the probe 106 is processed. In some instances, the image data may be processed by the AI circuit 120.

At step 520, an input regarding the ultrasound imaging workflow is received. The input refers to a request for help/assistance/guidance from the chatbot regarding the ultrasound imaging workflow. The input may be a text input, a voice input, or a selection of a pre-defined prompt. The text input may be received via a text-box presented to the user via the display device 132. In some embodiments, and as shown below with reference to FIGS. 7-8, the text input may be entered as a free-text entry into a text-box included in the virtual assistance window 710. For example, if the input from the user is “Help me improve my exam,” the user may type “Help me improve my exam” into the text-box of the virtual assistance window 710. The voice input may be received via the microphone in the user interface 130. Continuing with the same example, if the input from the user is “Help me improve my exam,” the user may speak the words “Help me improve my exam” while within a range (e.g., a few feet, a few yards, within a same room, etc.) of the user interface 130.

In some embodiments, the display device 132 may present a plurality of pre-defined prompts to the user of the ultrasound imaging system 100. The pre-defined prompts refer to buttons or other selectable elements configured to provide an instruction, prompt, input, etc. to the chatbot when selected (e.g., clicked on, pressed, etc.) by a user. In some instances, the plurality of pre-defined prompts may include universal prompts presented via any environment in which ultrasound imaging system 100 is being used, to any user of the ultrasound imaging system 100, and/or during any type of procedure being performed by the ultrasound imaging system 100. For example, the input “Help me improve my exam” may be a universal prompt represented by a selectable element (e.g., a “Help me improve my exam” button) on the display device 132.

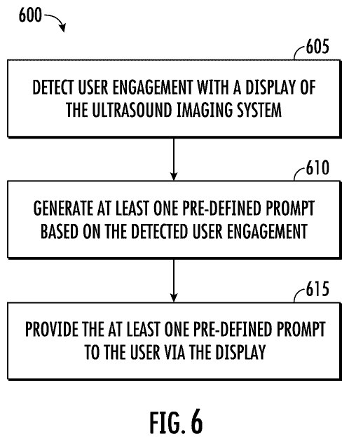

Alternatively or additionally, at least one of the plurality of pre-defined prompts may be unique to the environment in which ultrasound imaging system 100 is being used, to the user of the ultrasound imaging system 100, and/or to the type of procedure being performed by the ultrasound imaging system 100. Referring to FIG. 6, a flow chart is shown illustrating a method 600 for generating the pre-defined prompts during the method 500. In such embodiments, the AI circuit 120 may be configured to generate the plurality of pre-defined prompts and present the plurality of pre-defined prompts to the user via the display device 132. Furthermore, in this way, the plurality of pre-defined prompts maybe generated and presented in real-time such that as a status/condition of the ultrasound imaging workflow changes, the pre-defined prompts presented to the user may also change.

As shown in FIG. 6, method 600 begins by detecting user engagement with the display device 132 (e.g., via GUI 700 and/or GUI 900, as described below with reference to FIGS. 7 and 9, respectively) at step 605. The user may be the operator of the ultrasound imaging system 100 (e.g., a sonographer performing an ultrasound scan). For example, if the user hovers a cursor over a specific location of an ultrasound image (e.g., represented by element 910 depicted over ultrasound image 905 on GUI 900, as described in greater detail below with reference to FIG. 9), the AI model 120 may detect such user engagement with the display device 132. In this way, the AI circuit 120 may consider contextual information (e.g., the detection of the interaction of the user with the display device 132) when generating the pre-defined prompts.

Based on the user engagement detected at step 605, method 600 continues with generating at least one pre-defined prompt at step 610. That is, continuing with the example provided above, the AI circuit 120 may generate one or more pre-defined prompts relating to the specific location being hovered over by the cursor. For instance, a pre-defined prompt may be “Detect any abnormalities in this region,” where the region refers to the specific location/area on the ultrasound image being hovered over by the cursor. At step 615, the at least one pre-defined prompt is provided to the user (e.g., the operator) via the display of the ultrasound imaging system 100.

Referring to FIG. 5, The input may be received at step 520 as an input to the chatbot provided at step 505. In other words, the input may be received as an input to the AI circuit 120 (e.g., the LLM 310 and/or the VLM 312).

After receiving the input at step 520, the method 500 includes identifying contextual information regarding the ultrasound imaging workflow at step 525. The contextual information identified at step 525 refers to a portion of contextual information from a database of contextual information relating to the ultrasound imaging system 100 (e.g., the database of contextual information 420, as described above with reference to FIGS. 4A and 4B). In some embodiments, the AI circuit 120 is configured to identify the portion of contextual information from the database of contextual information at step 525.

Based on the contextual information identified at step 525 and the image data processed at step 515, method 500 continues with generating a response to the input at step 530. The response may be generated by the AI circuit 120.

At step 535, the response generated at step 530 is provided to the user. The response may be provided as an output from the chatbot by the AI circuit 120 (e.g., as shown by the chatbot assistance window 710 in FIG. 8). In some embodiments, providing the response to the input includes at least one of providing a textual response, providing an audible response, or changing an operating characteristic of the ultrasound imaging system 100. The textual response may be provided as a textual output via the chatbot assistance window 710, as described below with reference to FIG. 8. The audible response may be provided to the user via a speaker within the ultrasound imaging system 100. In various instances, changing the operating characteristic of the ultrasound imaging system 100 includes changing at least one of a mode (e.g., B-mode, color flow Doppler mode, M-mode, color M-mode, spectral Doppler, elastography, TVI, strain, strain rate, etc.), a view, an acquisition parameter, or a measurement setting of the ultrasound imaging system 100.

Referring to FIG. 7, a GUI 700 displaying an ultrasound image 705 of an anatomical structure taken during an ultrasound examination (e.g., an echocardiogram) is shown. In some embodiments, the GUI 700 may be displayed via the display device 132 of the ultrasound imaging system 100. The ultrasound image 705 may be obtained by the ultrasound imaging system 100 shown by FIG. 1 and described above. In some embodiments, the ultrasound image 705 may be one image of a group of images obtained sequentially by probe 106 while operating in a specific mode (e.g., the B-mode). The ultrasound image 705 may be a static image or may be a series of images (e.g., video feed showing a cine loop). According to certain implementations, the ultrasound image 705 displayed on the GUI 700 may update in real-time as the ultrasound examination occurs and as more ultrasound data is collected by the probe 106.

The GUI 700 is also shown to include the chatbot assistance window 710. The chatbot assistance window 710 refers to an interface element by which a user (e.g., sonographer) of the ultrasound imaging system may interact with the chatbot described herein. As shown, the chatbot assistance window 710 includes a free-text box in which the user can submit an input to the chatbot (e.g., a question, a request for guidance, an instruction to change an operating characteristic, etc.). For example, the chatbot assistance window 710 provides example queries “What can I use the MVQ tool for?” and “What measurements are supported by 4D Auto LVQ?”

As shown in FIG. 7, the GUI 700 may include a display of imaging parameters 715. The imaging parameters 715 refer to imaging parameters of the ultrasound imaging system 100 applied during acquisition of the ultrasound image 705. In some embodiments, the imaging parameters 715 may be default imaging parameters (e.g., unadjusted imaging parameters) associated with a mode from which the ultrasound image 705 was captured. For example, and as shown, the imaging parameters may include a frame rate (FPS) (e.g., 70 frames per second), a frequency (e.g., 1.7/3.3 MHz), a power (e.g., 0 dB), a gain (e.g., 0 dB), a compression (e.g., 60 dB), a persistence (e.g., 1.4), and a depth (e.g., 16.0 cm) applied during the acquisition of the ultrasound image 705.

Referring to FIG. 8, an illustration of a process for generating and providing the chatbot response during method 500 using the GUI 700 is shown. As shown, the process begins with a prompt/task 805. For example, the prompt/task 805 may be: “I would like to only measure E′, A′, and S′”. The chatbot receives the prompt/task 805 and identifies the interface 810 with which the prompt/task 805 may be answered. That is, because the prompt/task 805 relates to measurement settings of the ultrasound imaging system 100, the chatbot identifies an interface by which as user can update measurement settings of the ultrasound imaging system 100 (e.g., the interface 810). The response generated by the chatbot is provided to the user via the chatbot assistance window 710 (e.g., displayed on the GUI 700, as shown in FIG. 7). For example, the response to the prompt/task 805 may be “In Config-Meas./Text you can disable the S′E′A′ combined measurement and enable the specific E′ measurement.”

Referring to FIG. 9, a GUI 900 including an ultrasound image 905 obtained via the ultrasound imaging system 100 is shown. In some embodiments, the GUI 900 may be displayed via the display device 132 of the ultrasound imaging system 100. The ultrasound image 905 may be obtained by the ultrasound imaging system 100 shown by FIG. 1 and described above. In some embodiments, the ultrasound image 905 may be one image of a group of images obtained sequentially by probe 106 while operating in a specific mode (e.g., the B-mode). The ultrasound image 905 may be a static image or may be a series of images (e.g., video feed showing a cine loop). According to certain implementations, the ultrasound image 905 displayed on the GUI 900 may update in real-time as the ultrasound examination occurs and as more ultrasound data is collected by the probe 106.

As shown in FIG. 9, the GUI 900 includes an element 910 depicted over the ultrasound image 905. As described above with reference to FIG. 6, the element 910 may represent a cursor hovering over a location/area of the ultrasound image 905. For example, after navigating the cursor (e.g., the element 910) to the desired location/area of the ultrasound image 905, the user of the ultrasound imaging system 100 may submit the question (e.g., as an input to the chatbot) “Is there anything abnormal in this region?” where “this region” refers to the location/area of the ultrasound image 905 being hovered over by the element 910. The question may be submitted as a text entry (e.g., via the chatbot assistance window 710), a voice entry, or a selection of a pre-defined prompt (e.g., a “detect abnormalities in this region” button). In response, the AI circuit 120 may identify the area over which the element 910 is currently hovering and may proceed with generating a response to the question including any detected abnormalities in that area.

The GUI 900 may also include a display of imaging parameters 915. The imaging parameters 915 refer to imaging parameters of the ultrasound imaging system 100 applied during acquisition of the ultrasound image 905. In some embodiments, the imaging parameters 915 may be default imaging parameters (e.g., unadjusted imaging parameters) associated with a mode from which the ultrasound image 905 was captured. For example, and as shown, the imaging parameters may include a frequency (e.g., 1.7/3.3 MHz), a power (P) (e.g., 0 dB), a gain (G(t) (e.g., −9 dB), a compression (Compr) (e.g., 50 dB), a persistence (Pers) (e.g., 0.2), and a depth (D) (e.g., 15.0 cm) applied during the acquisition of the ultrasound image 905.

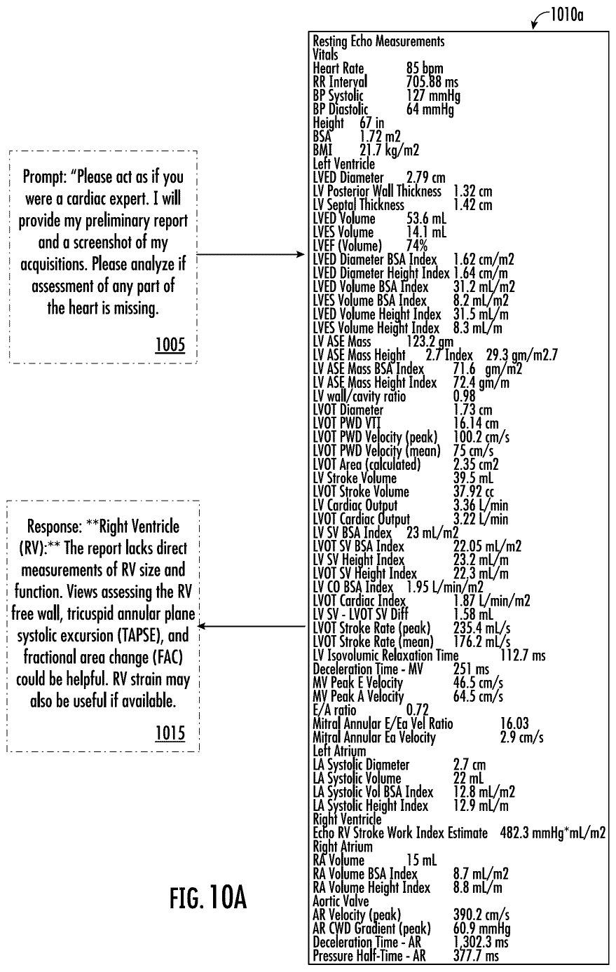

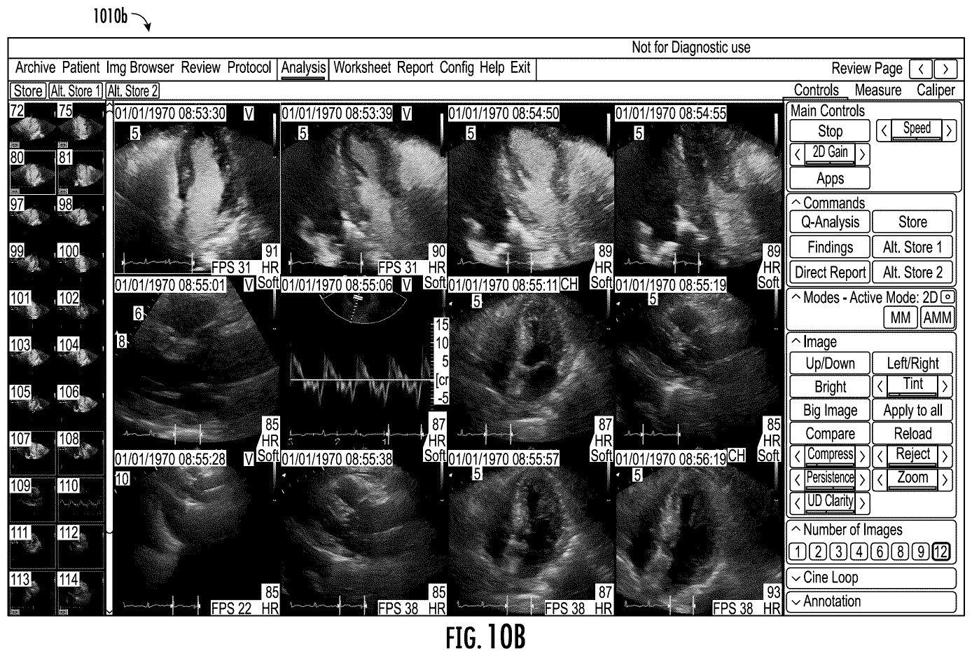

Referring to FIGS. 10A and 10B, contextual information used to provide chatbot assistance during the method 500 is shown. FIG. 10A depicts a textual illustration 1010a of the contextual information, while FIG. 10B depicts a graphical illustration 1010b of the contextual information. As shown, the contextual information illustrated by FIGS. 10A and 10B may be identified in response to a prompt 1005 of “Please act as if you were a cardiac expert. I will provide my preliminary report and a screenshot of my acquisitions. Please analyze if assessment of any part of the heart is missing.” In this example, the textual illustration 1010a of the contextual information may be the preliminary report, while the graphical illustration 1010b of the contextual information may be the screenshots. Based on the contextual information shown in FIGS. 10A and 10B, the chatbot may provide a response 1015 of “**Right Ventricle (RV): ** The report lacks direct measurements of RV size and function. Views assessing the RV free wall, tricuspid annular plane systolic excursion (TAPSE), and fractional area change (FAC) could be helpful. RV strain may also be useful if available.”

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

What is claimed is:

1. An ultrasound imaging system comprising:
a transducer configured to transmit and receive an ultrasound signal;
a matching layer configured to have an acoustic impedance between a tissue to be imaged and a material of the transducer;
a damping block configured to absorb ultrasound energy; and
an artificial intelligence (AI) processing circuit configured to:
receive image data obtained by the transducer;
generate a natural language input based on the image data, the natural language input configured to prompt a response from the AI processing circuit based on the natural language input;
input the natural language input to a machine learning model during an ultrasound imaging workflow performed by the ultrasound imaging system;
identify, from a database of contextual information relating to the ultrasound imaging system, a portion of the contextual information regarding the ultrasound imaging workflow;
generate the response based on the portion of the contextual information and the processed image data; and
provide, to a user via a display of the ultrasound imaging system, the response.

2. The ultrasound imaging system of claim 1, wherein the AI processing circuit is further configured to receive at least one of a text input or a voice input from the user, wherein the at least one of the text input or the voice input is configured to prompt the response from a chatbot generated by AI processing circuit.

3. The ultrasound imaging system of claim 1, wherein the AI processing circuit is configured to:
encode a selectable element with the natural language input;
present, to the user via the display, the selectable element encoded with the natural language input; and
receive a selection of the selectable element from the user, wherein the selection of the selectable element is configured to input the natural language input to the machine learning model.

4. The ultrasound imaging system of claim 3, wherein the selectable element is one of a plurality of selectable elements each encoded with one of a plurality of natural language inputs, and wherein the AI processing circuit is configured to:
generate the plurality of selectable elements; and
present the plurality of selectable elements to the user via the display of the ultrasound imaging system.

5. The ultrasound imaging system of claim 4, wherein at least one of the plurality of selectable elements is generated based on the AI processing circuit detecting an interaction of the user with the display of the ultrasound imaging system.

6. The ultrasound imaging system of claim 1, wherein providing the response comprises at least one of providing a textual response via a chatbot, providing an audible response, or changing an operating characteristic of the ultrasound imaging system.

7. The ultrasound imaging system of claim 6, wherein changing the operating characteristic of the ultrasound imaging system comprises changing at least one of a mode, a view, an acquisition parameter, or a measurement setting.

8. The ultrasound imaging system of claim 1, wherein the AI processing circuit comprises at least one of a large language model or a visual language model.

9. The ultrasound imaging system of claim 8, wherein the AI processing circuit comprises a retrieval-augmented generation model and wherein generating the response comprises:
querying a retrieval model configured to search a medical information database for data relating to the ultrasound imaging workflow;
combining the data relating to the ultrasound imaging workflow with the natural language input to create an augmented prompt;
applying the augmented prompt to the at least one of the large language model or the visual language model; and
generating the response based on the augmented prompt.

10. The ultrasound imaging system of claim 1, wherein the database of contextual information comprises at least one of a real-time status of the ultrasound imaging workflow, a current configuration of the display of the ultrasound imaging system, patient medical history, a user guide associated with the ultrasound imaging system, or a medical information database.

11. An ultrasound imaging system comprising:
a transducer configured to transmit and receive an ultrasound signal;
a matching layer configured to have an acoustic impedance between a tissue to be imaged and a material of the transducer;
a damping block configured to absorb ultrasound energy; and
a processing circuit comprising a processor coupled to a memory device storing instructions thereon that, when executed, cause the processing circuit to perform operations comprising:
receiving, by an artificial intelligence (AI) processing circuit, image data obtained by the transducer;
generating a natural language input based on the image data, the natural language input configured to prompt a response from the AI processing circuit based on the natural language input;
inputting the natural language input to a machine learning model during an ultrasound imaging workflow performed by the ultrasound imaging system;
identifying, from a database of contextual information relating to the ultrasound imaging system, a portion of the contextual information related to the ultrasound imaging workflow;
generating, using the AI processing circuit, the response based on the portion of the contextual information and the processed image data; and
providing, to a user via a display of the ultrasound imaging system, the response.

12. The ultrasound imaging system of claim 11, wherein the operations further comprise receiving at least one of a text input or a voice input from the user, wherein the at least one of the text input or the voice input is configured to prompt the response from a chatbot generated by the AI processing circuit.

13. The ultrasound imaging system of claim 11, wherein the operations comprise:
encoding a selectable element with the natural language input;
presenting, to the user via the display, the selectable element encoded with the natural language input; and
receiving a selection of the selectable element from the user, wherein the selection of the selectable element is configured to input the natural language input to the machine learning model.

14. The ultrasound imaging system of claim 13, wherein the selectable element is one of a plurality of selectable elements each encoded with one of a plurality of natural language inputs, and wherein the operations comprise:
generating the plurality of selectable elements; and
presenting the plurality of selectable elements to the user via the display of the ultrasound imaging system.

15. The ultrasound imaging system of claim 11, wherein the AI processing circuit comprises a retrieval-augmented generation model and at least one of a large language model or a visual language model, and wherein generating the response comprises:
querying, by the AI processing circuit, a retrieval model configured to search a medical information database for data relating to the ultrasound imaging workflow;
combining, by the AI processing circuit, the data relating to the ultrasound imaging workflow with the natural language input to create an augmented prompt;
applying, by the AI processing circuit, the augmented prompt to the at least one of the large language model or the visual language model; and
generating, by the AI processing circuit, the response based on the augmented prompt.

16. A method comprising:
receiving, by an artificial intelligence (AI) processing circuit, image data obtained by an ultrasound probe of an ultrasound imaging system;
generating, by the AI processing circuit, a natural language input based on the image data, the natural language input configured to prompt a response from the AI processing circuit based on the natural language input;
inputting, by the AI processing circuit, the natural language input to a machine learning model during an ultrasound imaging workflow performed by the ultrasound imaging system;
identifying, by the AI processing circuit and from a database of contextual information relating to the ultrasound imaging system, a portion of the contextual information regarding the ultrasound imaging workflow;
generating, by the AI processing circuit, the response based on the portion of the contextual information and the processed image data; and
providing, by the AI processing circuit and to a user via a display, the response.

17. The method of claim 16, further comprising receiving, by the AI processing circuit, at least one of a text input or a voice input from the user, wherein the at least one of the text input or the voice input is configured to prompt the response from a chatbot generated by the AI processing circuit.

18. The method of claim 16, wherein the method comprises:
encoding, by the AI processing circuit, a selectable element with the natural language input;
presenting, by the AI processing circuit and to the user via the display, the selectable element encoded with the natural language prompt; and
receiving, by the AI processing circuit a selection of the selectable element from the user, wherein the selection of the selectable element is configured to input the natural language prompt to the machine learning model.

19. The method of claim 16, wherein the AI processing circuit comprises a retrieval-augmented generation model and at least one of a large language model or a visual language model, and wherein generating the response comprises:
querying, by the AI processing circuit, a retrieval model configured to search a medical information database for data relating to the ultrasound imaging workflow;
combining, by the AI processing circuit, the data relating to the ultrasound imaging workflow with the natural language input to create an augmented prompt;
applying, by the AI processing circuit, the augmented prompt to the at least one of the large language model or the visual language model; and
generating, by the AI processing circuit, the response based on the augmented prompt.

20. The method of claim 16, wherein providing the response comprises at least one of providing a textual response via a chatbot, providing an audible response, or changing an operating characteristic of the ultrasound imaging system, and wherein changing the operating characteristic of the ultrasound imaging system comprises changing at least one of a mode, a view, an acquisition parameter, or a measurement setting.
